(19) United States
(12) Patent Application Publication
(10) Pub. No.: US 2026/0115183 A1
(43) Pub. Date: Apr. 30, 2026

(54) METHOD AND COMPOSITION FOR PROTECTING OR IMPROVING OVARIAN FUNCTION IN MAMMALS

(71) Applicant: NANJING NUTRABUILDING BIO-TECH CO., LTD., Nanjing (CN)

(72) Inventors: Ronghua YI, Nanjing (CN); Kylin LIAO, Nanjing (CN); Shawn WELLS, Frisco, TX (US)

(21) Appl. No.: 19/432,851

(22) Filed: Dec. 24, 2025

Related U.S. Application Data
(63) Continuation of application No. PCT/CN2024/101298, filed on Jun. 25, 2024.

(30) Foreign Application Priority Data
Jun. 28, 2023 (WO) PCT/CN2023/103075

(51) Int. Cl. A61K 31/4745; A61K 31/047; A61K 31/4172; A61K 36/185; A61P 15/08; A61P 15/12

(52) U.S. Cl. CPC A61K 31/4745; A61K 31/047; A61K 31/4172; A61K 36/185; A61P 15/08; A61P 15/12

(57) ABSTRACT

The invention discloses method and composition for protecting or improving ovarian function in mammals and related applications thereof in preparing foods, drinks, nutritional supplements, animal feeds for protecting or improving ovarian function in mammals. The method and composition of the invention can improve the quantity and quality of follicles, improve ovarian function and delay aging.

### CROSS REFERENCE TO RELATED APPLICATION

This application is a continuation application of International Patent Application No. PCT/CN2024/101298, filed on Jun. 25, 2024, which claims the priority of the International Application No. PCT/CN2023/103075, filed on Jun. 28, 2023, the contents of all of which are incorporated herein by reference in their entirety.

### FIELD OF THE INVENTION

This invention belongs to the technical field of dietary or nutritional supplements; specifically relates to method and composition for protecting or improving ovarian function in mammals and related applications thereof in preparing foods, drinks, nutritional supplements, animal feeds for protecting or improving ovarian function in mammals.

### BACKGROUND OF THE INVENTION

There are tens of thousands of follicles in the female ovary. The main function of the ovary is ovulation and secretion of female hormones. From puberty, the ovary ovulates periodically. Ovarian aging refers to the process that women's reserves gradually decline until failure with age, which is based on the decline of follicular number and egg quality, and finally manifests as sterilization and even menopause, and affects many systems and organs, leading to the occurrence and development of related diseases and syndromes.

Women's ovarian function begins to decline around 45-50 years old, and their estrogen level drops, and they enter perimenopausal period. However, some women before the age of 40 will have premature ovarian failure due to factors such as environment, diet, bad living habits, psychology or pathology. Ovarian aging will cause women's physical and physiological changes, such as menstrual changes, osteoporosis, increased blood pressure, increased skin wrinkles or menopausal symptoms such as hot flashes, emotional instability and irritability, and the body's immunity will decline, which will accelerate aging and seriously affect women's quality of life.

Ovarian aging is a complex biological process in which many factors interact and accumulate gradually. With the passage of years, follicles are constantly consumed, and the decrease in the number of follicles is the main cause of ovarian aging. However, the accumulation of metabolites in the body, the changes of microenvironment in the ovary, such as the damage of free radicals and advanced glycation end products to follicles, as well as mitochondrial DNA mutation, telomere shortening and other factors, lead to the decrease of the quality of the remaining follicles at the same time. With the accelerated pace of social life and the great changes in the economic and demographic situation, the diseases related to ovarian aging in women have obviously increased, and the social problems brought about by ovarian aging have gradually become prominent, so the diseases and problems related to ovarian aging have become a social problem of universal concern.

At present, there are mainly ways to improve and treat ovarian aging and its related symptoms and diseases: Hormone replacement therapy (HRT), but the existing research clearly shows that it has many side effects and long-term carcinogenicity, which obviously limits its clinical application. Traditional Chinese medicine advocates “tonifying kidney and nourishing Yin, soothing liver and nourishing blood” to delay ovarian aging, but at present, there may be misunderstandings of indiscriminate medication and treatment in traditional Chinese medicine prescriptions without understanding its etiology and pathology. In addition, there are few kinds of foods or health foods that can improve polycystic ovary syndrome or delay ovarian aging in the market, among which soybean isoflavones are the main components, and the components are simple and the therapeutic effect is not ideal.

Ergothioneine (EGT) is a safe natural compound with excellent anti-inflammatory and antioxidant properties. Pyrroloquinoline quinone (PQQ) is a small molecule which is soluble in water and has good thermal stability. Mammals themselves can't synthesize PQQ, but they can get it from food, such as vegetables and meat. PQQ is an indispensable nutrient for maintaining the health of mammals, and is known as a longevity factor. It is very important to study the relationship of ergothioneine (EGT) and PQQ with ovarian function.

### SUMMARY OF THE INVENTION

To achieve the above object, in one aspect, the present invention provides a method for protecting or improving ovarian function in a mammal, wherein the method comprises administering to the mammal a composition comprising an effective amount of ergothioneine or a pharmaceutically acceptable salt, acid, ester, analog or derivative thereof. In some embodiments, the composition may further comprise a pharmaceutically acceptable carrier.

In some embodiments, the composition further comprises an effective amount of Pyrroloquinoline quinone or a pharmaceutically acceptable salt, acid, ester, analog or derivative thereof.

In some embodiments, the composition further comprises an effective amount of Oenothera biennis extract. The Oenothera biennis extract can be Oenothera biennis oil, for example.

In some embodiments, the composition further comprises an effective amount of inositol.

In some embodiments, the protecting or improving ovarian function in a mammal includes promoting follicular development, increasing the number of follicles or improving the quality of follicles.

In some embodiments, the method is used for delaying or improving premature ovarian failure or aging, improving polycystic ovary syndrome, relieving dysfunctional uterine bleeding, delaying menopause and/or relieving menopausal symptoms.

In some embodiments, the composition is formulated as suppository, tablet, pill, granule, powder, film, injection, capsule, aerosol, elixir, tincture, tonic, solution, liquid suspension or syrup.

In some embodiments, the composition is prepared into food, beverage, nutritional supplement, animal feed.

In some embodiments, the composition is administered by oral administration, intravenous injection, intramuscular injection, intraperitoneal or sublingual administration.

In some embodiments, the ergothioneine or the pharmaceutically acceptable salt, acid, ester, analog or derivative thereof is formulated to be administered in an amount of 1-1500 mg per day. In some embodiments, the ergothioneine or the pharmaceutically acceptable salt, acid, ester, analog or derivative thereof may be formulated to be administered in an amount of 1-1000 mg, 2-500 mg, 3-200 mg, 4-100 mg, 5-50 mg, 10-30 mg per day.

In some embodiments, the Pyrroloquinoline quinone or the pharmaceutically acceptable salt, acid, ester, analog or derivative thereof is formulated to be administered in an amount of 1-1000 mg per day. In some embodiments, the Pyrroloquinoline quinone or the pharmaceutically acceptable salt, acid, ester, analog or derivative thereof may be formulated to be administered in an amount of 2-500 mg, 3-200 mg, 5-100 mg, 10-50 mg, 15-40 mg per day.

In some embodiments, the Oenothera biennis extract is formulated to be administered in an amount of 10-2000 mg per day. In some embodiments, the Oenothera biennis extract may be formulated to be administered in an amount of 15-1500 mg, 20-1000 mg, 50-800 mg, 100-600 mg, 200-500 mg per day.

In some embodiments, the inositol is formulated to be administered in an amount of 10-3000 mg per day. In some embodiments, the inositol may be formulated to be administered in an amount of 15-2000 mg, 20-1500 mg, 50-1000 mg, 80-500 mg, 100-400 mg per day.

In some embodiments, the weight ratio of ergothioneine, Pyrroloquinoline quinone, Oenothera biennis extract and inositol is (1-10):(1-10):(10-100):(80-400). In some embodiments, the weight ratio of ergothioneine, Pyrroloquinoline quinone, Oenothera biennis extract and inositol may be (1-5):(1-4):(20-80):(80-400), 1:1:20:80, 1:1:40:100, 1:1:40:160, 1:1:60:200, 5:4:80:400, 5:4:20:100, 5:4:20:80.

In another aspect, the present invention provides a composition comprising ergothioneine or a pharmaceutically acceptable salt, acid, ester, analog or derivative thereof, for protecting or improving ovarian function in a mammal. In some embodiments, the composition may further comprise a pharmaceutically acceptable carrier.

In some embodiments, the composition further comprises an effective amount of Pyrroloquinoline quinone or a pharmaceutically acceptable salt, acid, ester, analog or derivative thereof.

In some embodiments, the composition further comprises an effective amount of Oenothera biennis extract. The Oenothera biennis extract can be Oenothera biennis oil, for example.

In some embodiments, the composition further comprises an effective amount of inositol.

In some embodiments, the protecting or improving ovarian function in a mammal includes promoting follicular development, increasing the number of follicles or improving the quality of follicles.

In some embodiments, the composition is used for delaying or improving premature ovarian failure or aging, improving polycystic ovary syndrome, relieving dysfunctional uterine bleeding, delaying menopause and/or relieving menopausal symptoms.

In some embodiments, the composition is formulated as suppository, tablet, pill, granule, powder, film, injection, capsule, aerosol, elixir, tincture, tonic, solution, liquid suspension or syrup.

In some embodiments, the composition is prepared into food, beverage, nutritional supplement, animal feed.

In some embodiments, the ergothioneine or the pharmaceutically acceptable salt, acid, ester, analog or derivative thereof is formulated to be administered in an amount of 1-1500 mg per day. In some embodiments, the ergothioneine or the pharmaceutically acceptable salt, acid, ester, analog or derivative thereof may be formulated to be administered in an amount of 1-1000 mg, 2-500 mg, 3-200 mg, 4-100 mg, 5-50 mg, 10-30 mg per day.

In some embodiments, the Pyrroloquinoline quinone or the pharmaceutically acceptable salt, acid, ester, analog or derivative thereof is formulated to be administered in an amount of 1-1000 mg per day. In some embodiments, the Pyrroloquinoline quinone or the pharmaceutically acceptable salt, acid, ester, analog or derivative thereof may be formulated to be administered in an amount of 2-500 mg, 3-200 mg, 5-100 mg, 10-50 mg, 15-40 mg per day.

In some embodiments, the Oenothera biennis extract is formulated to be administered in an amount of 10-2000 mg per day. In some embodiments, the Oenothera biennis extract may be formulated to be administered in an amount of 15-1500 mg, 20-1000 mg, 50-800 mg, 100-600 mg, 200-500 mg per day.

In some embodiments, the inositol is formulated to be administered in an amount of 10-3000 mg per day. In some embodiments, the inositol may be formulated to be administered in an amount of 15-2000 mg, 20-1500 mg, 50-1000 mg, 80-500 mg, 100-400 mg per day.

In another aspect, the present invention provides use of ergothioneine or a physiologically acceptable salt, acid, ester, analog or derivative thereof in the preparation of a composition for protecting or improving ovarian function in a mammal. In some embodiments, the composition may further comprise a pharmaceutically acceptable carrier.

In some embodiments, the composition further comprises an effective amount of Pyrroloquinoline quinone or a pharmaceutically acceptable salt, acid, ester, analog or derivative thereof.

In some embodiments, the composition further comprises an effective amount of Oenothera biennis extract. The Oenothera biennis extract can be Oenothera biennis oil, for example.

In some embodiments, the composition further comprises an effective amount of inositol.

In some embodiments, the protecting or improving ovarian function in a mammal includes promoting follicular development, increasing the number of follicles or improving the quality of follicles.

In some embodiments, the composition is used for delaying or improving premature ovarian failure or aging, improving polycystic ovary syndrome, relieving dysfunctional uterine bleeding, delaying menopause and/or relieving menopausal symptoms.

In some embodiments, the composition is formulated as suppository, tablet, pill, granule, powder, film, injection, capsule, aerosol, elixir, tincture, tonic, solution, liquid suspension or syrup.

In some embodiments, the ergothioneine or the pharmaceutically acceptable salt, acid, ester, analog or derivative thereof is formulated to be administered in an amount of 1-1500 mg per day. In some embodiments, the ergothioneine or the pharmaceutically acceptable salt, acid, ester, analog or derivative thereof may be formulated to be administered in an amount of 1-1000 mg, 2-500 mg, 3-200 mg, 4-100 mg, 5-50 mg, 10-30 mg per day.

In some embodiments, the Pyrroloquinoline quinone or the pharmaceutically acceptable salt, acid, ester, analog or derivative thereof is formulated to be administered in an amount of 1-1000 mg per day. In some embodiments, the Pyrroloquinoline quinone or the pharmaceutically acceptable salt, acid, ester, analog or derivative thereof may be formulated to be administered in an amount of 2-500 mg, 3-200 mg, 5-100 mg, 10-50 mg, 15-40 mg per day.

In some embodiments, the Oenothera biennis extract is formulated to be administered in an amount of 10-2000 mg per day. In some embodiments, the Oenothera biennis extract may be formulated to be administered in an amount of 15-1500 mg, 20-1000 mg, 50-800 mg, 100-600 mg, 200-500 mg per day.

In some embodiments, the inositol is formulated to be administered in an amount of 10-3000 mg per day. In some embodiments, the inositol may be formulated to be administered in an amount of 15-2000 mg, 20-1500 mg, 50-1000 mg, 80-500 mg, 100-400 mg per day.

In another aspect, the present invention provides a method for managing glucose tolerance or reducing fasting blood glucose level in an individual, wherein the method comprises administering to the mammal a composition comprising an effective amount of ergothioneine or a pharmaceutically acceptable salt, acid, ester, analog or derivative thereof.

In some embodiments, the composition further comprises an effective amount of Pyrroloquinoline quinone or a pharmaceutically acceptable salt, acid, ester, analog or derivative thereof.

In some embodiments, the composition further comprises an effective amount of Oenothera biennis extract. The Oenothera biennis extract can be Oenothera biennis oil.

In some embodiments, the composition further comprises an effective amount of inositol.

This summary is provided to introduce the selection of concepts in a simplified form, which are further described in the detailed description below. Summary is not intended to identify key features or essential features of the claimed subject matter, nor is it intended to be used to limit the scope of the claimed subject matter.

### BRIEF DESCRIPTION OF THE DRAWINGS

FIG. 1 is a graph showing total testosterone T in each experimental group of the present invention.

FIG. 2 is a graph showing estradiol E2 in each experimental group of the present invention.

FIG. 3 is a graph showing luteinizing hormone LH in each experimental group of the present invention.

FIG. 4 is a graph showing follicle stimulating hormone FSH in each experimental group of the present invention.

FIG. 5 is a graph showing the changes of blood glucose in each experimental group of the present invention.

FIG. 6 is a graph showing HMOA-IR in each experimental group of the present invention.

FIG. 7 is a graph showing total cholesterol TC in each experimental group of the present invention.

FIG. 8 is a graph showing triglyceride TG in each experimental group of the present invention.

### DETAILED DESCRIPTION OF THE INVENTION

Reference will now be made in detail to the preferred embodiments of the present disclosure, examples of which are further illustrated. While the present disclosure will be described in conjunction with the preferred embodiments, it will be understood that they are not intended to limit the present disclosure to these embodiments. To the contrary, the present disclosure is intended to cover alternatives, modifications and equivalents, which may be included within the spirit and scope of the present disclosure as defined by the claims.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” or their conjugations, refer to a situation where said terms are used in their non-limiting sense to mean that items following the word are included, but items not specifically mentioned are not excluded. It also encompasses the more limiting verb ‘to consist essentially of’ and ‘to consist of’.

As used herein, the term “mammal” or “subject” may be used interchangeably to refer to any animal to which the presently disclosed methods and compositions may be applied or administered. The animal may have an illness or other disease, but the animal does not need to be sick to benefit from the presently disclosed methods and compositions. As such, any animal may apply the disclosed compositions, or be a recipient of the disclosed methods. Although the animal subject is preferably a human, the methods and compositions of the present disclosure have application in veterinary medicine as well, e.g., for the treatment of domesticated species such as canine, feline, murine, and various other pets; farm animal species such as bovine, equine, ovine, caprine, porcine, etc.; and wild animals, e.g., in the wild or in a zoological garden, such as non-human primates.

As used herein, the term “administration” refers to the process of delivering a disclosed composition or active ingredient to a subject. The composition of the present invention is preferably administered by oral administration, intravenous injection, intramuscular injection, intraperitoneal or sublingual administration, etc., but it can also be administered by other conventional routes to exert the desired effect.

As used herein, the term “effective amount” refers to the amount required to achieve an effect as taught herein. Effective amount herein includes, but is not limited to, amount necessary to protect or improve ovarian function in mammals, and/or amount necessary to promote follicular development, increase the number of follicles, improve the quality of follicles, and/or amount necessary to delay or improve premature ovarian failure or aging, improve polycystic ovary syndrome, relieve dysfunctional uterine bleeding, delay menopause and/or alleviate menopausal symptoms. In accordance with the present disclosure, a suitable single dose size is a dose that is capable of achieve the above effects when administered one or more times over a suitable time period.

As used herein, the term “pharmaceutically acceptable” means pharmaceutically, physiologically, alimentarily acceptable, and refers to those compositions or combinations of agents, materials, or compositions, and/or their dosage forms, which are within the scope of sound medical judgment, suitable for use in contact with the tissues of human beings and animals, compatible with other components of the composition, without excessive toxicity, irritation, allergic response, or other problem or complication, commensurate with a reasonable benefit/risk ratio.

In some embodiments, ergothioneine or a pharmaceutically acceptable salt, acid, ester, analog or derivative thereof and/or the composition comprising the same of the present invention can be prepared into a composition together with a dietetically or pharmaceutically acceptable carrier. The above carriers include non-toxic and compatible substances commonly used in health foods, dietary supplements and pharmaceutical preparations, such as sugar, starch, cellulose and its derivatives, powdered tragacanth, malt, gelatin, talc, oil, diol, polyol, ester, agar, alginic acid, pyrogen-free water, isotonic saline, etc.

In some embodiments, ergothioneine or a pharmaceutically acceptable salt, acid, ester, analog or derivative thereof and/or the composition comprising the same of the present invention can be administered together with other supplements, such as vitamins, minerals, tonics and other supplements known in the art.

The method of the present invention includes administering at least 1 mg, typically 1-1500 mg, preferably 1-1000 mg, 2-500 mg, 3-200 mg, 4-100 mg, 5-50 mg, 10-30 mg of ergothioneine or a pharmaceutically acceptable salt, acid, ester, analog or derivative thereof every day, depending on the specific preparation and form. The amount to be administered can also vary according to factors such as sensitivity, age, sex and weight of the subject, specific reaction, etc. One or more doses can be administered once or more times a day or at a suitable frequency for any period of time. For example, an effective dose can be administered daily for one day, several days, many days or indefinitely.

The composition of the present invention is simple and convenient to prepare, and can be prepared by uniformly mixing all the raw materials. In some embodiments, the preparation method of the composition of the present invention may include the following steps: firstly, uniformly mixing some raw materials, and then mixing them with other raw materials. More preferably, the raw materials are mixed in a three-dimensional way for 20-30 minutes. In some embodiments, various components in the composition of the present invention can each be dissolved in a suitable solvent to form a corresponding solution and/or suspension respectively, and then the solutions and/or suspensions of the various components are mixed to form a mixed form of the composition of the present invention dissolved in a solvent. In the preparation of the composition, various components can be reasonably added according to the properties of the raw materials by using a mixing method well known in the art.

The following examples are illustrative of selected embodiments of the present disclosure and are not meant to limit the scope of the present disclosure. Unless otherwise specified, the materials and reagents described in the following examples are all common commercial products and can be purchased in the market.

### Example 1

20 parts of ergothioneine and 20 parts of PQQ were evenly mixed in a three-dimensional biconical mixer, and then evenly mixed with 400 parts of inositol and 1,600 parts of Oenothera biennis extract to obtain the composition of Example 1.

### Example 2

20 parts of ergothioneine and 20 parts of PQQ were evenly mixed in a three-dimensional biconical mixer, and then evenly mixed with 800 parts of inositol and 2,000 parts of Oenothera biennis extract to obtain the composition of Example 2.

### Example 3

20 parts of ergothioneine and 20 parts of PQQ were evenly mixed in a three-dimensional biconical mixer, and then evenly mixed with 1,200 parts of inositol and 4,000 parts of Oenothera biennis extract to obtain the composition of Example 3.

### Example 4

25 parts of ergothioneine and 20 parts of PQQ were evenly mixed in a three-dimensional biconical mixer, and then evenly mixed with 400 parts of inositol and 2,000 parts of Oenothera biennis extract to obtain the composition of Example 4.

### Example 5

25 parts of ergothioneine and 20 parts of PQQ were evenly mixed in a three-dimensional biconical mixer, and then evenly mixed with 100 parts of inositol and 400 parts of Oenothera biennis extract to obtain the composition of Example 5.

### Example 6

Female Wistar rats weighing 220-230 g were selected, and all rats were free to eat. One week later, without any intervention, they were randomly divided into control group CK (n=10; Placebo); Model group MD (n=10; Placebo); Experimental group A (n=10; Ergothioneine 2.25 mg/kg/d); Experimental group B (n=10; Ergothioneine 2.25 mg/kg/d+Pyrroloquinoline quinone 1.8 mg/kg/d); Experimental group C (n=10; Ergothioneine 2.25 mg/kg/d+Pyrroloquinoline quinone 1.8 mg/kg/d+Oenothera biennis 18 mg/kg/d+inositol 9 mg/kg/d). Four groups of rats were injected with testosterone propionate (10 mg/kg, once a day for 6 weeks) and olive oil continuously. Administration was performed by gavage.

Gavage intervention in each group lasted for 8 weeks. Finally, the rats were anesthetized with 1% pentobarbital sodium, and the rats were killed when they were estrus after fasting for 16 h. Blood was collected through abdominal aorta, and the serum was taken after centrifugation at 4° C. for 15 min at a speed of 1000 g.

Hormone determination: Serum total testosterone T, luteinizing hormone LH, follicle stimulating hormone FSH and estradiol E2 were determined by ELISA. All the operations of Elisa kit were strictly in accordance with the instructions.

OGTT, insulin, HOMA-IR and blood lipid were measured in each group at the last week of gavage. After fasting for 16 h, fasting blood glucose (0 min) was measured. Subsequently, rats were intraperitoneally injected with 50% glucose solution (2 g/kg). At each time point (0 min, 30 min, 60 min, 120 min), the blood glucose of rats was measured by glucose oxidase method. Blood was taken from the tip of the tail for OGTT. ELISA kit and HOMA-IR formula [HOMA-IR=fasting insulin (mIU/L)×fasting blood glucose (mmol/L)/22.5] were used. Serum total cholesterol (TC) and total triglyceride (TG) were detected by total cholesterol detection kit and triglyceride detection kit.

Graphpad 8.0 software was used to analyze the data, and the result data was expressed as standard deviation (x±s), and the significance was analyzed by one-way ANOVA test.

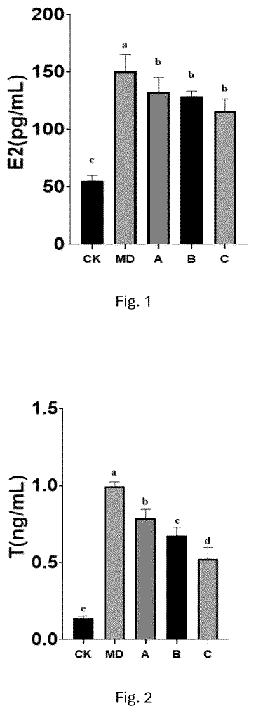

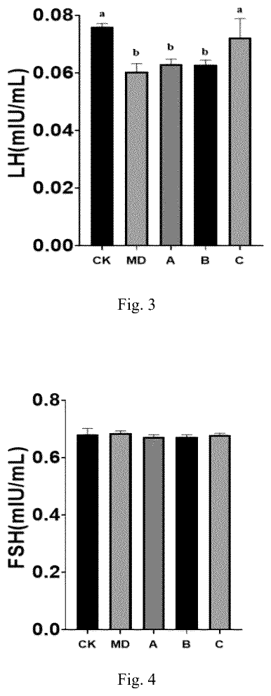

As shown in FIG. 1 to FIG. 4, after 6 weeks of testosterone propionate administration, the serum E2 and T levels of rats were significantly increased, LH levels were significantly decreased, and FSH levels were not significantly changed. After 8 weeks of intervention, compared with the Model group, the serum E2 and T of rats in Experimental groups A, B and C decreased significantly, and the serum E2 and T in Experimental group C decreased to the greatest extent, accounting for 22.9% and 47.3% respectively. At the level of serum T, the Experimental group C also showed significant difference over Experimental group A and Experimental group B. Compared with the Model group, only the Experimental group C showed a significant rebound in the serum LH level, which was 10%, and there was no significant difference between its serum LH level and that of the normal rats statistically. The results show that the Experimental group C (Ergothioneine+Pyrroloquinoline quinone+Oenothera biennis+inositol) has a positive regulatory effect on the hormone levels of rats.

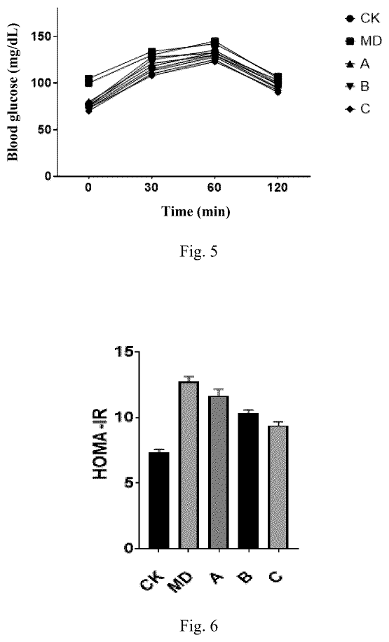

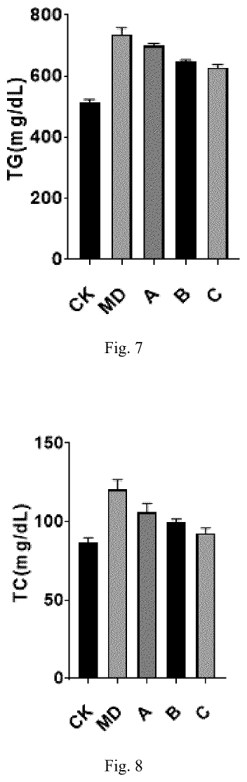

Glucose tolerance and blood lipid results: In the last week, OGTT was performed. Glucose gavage had no significant effect on the normal control group, and glucose tolerance showed normal. Compared with the normal control group, the glucose tolerance of the Model group animals increased. As shown in FIG. 5 to FIG. 8, compared with the control group, the blood glucose level of the Model group is higher at 30, 60 and 120 min. In addition, compared with the Model group, the blood glucose levels of animals in Experimental group A, Experimental group B and Experimental group C decreased significantly at different time points within 120 min, in which the initial blood glucose of Experimental group C was lower than that of Model group by 30 mg/dL, and the peak blood glucose at 60 min was lower than that of Model group by 20 mg/dL. The results show that the Experimental group C has the best effect on improving glucose tolerance. According to fasting blood glucose and fasting insulin, the HMOA-IR index was calculated. The HMOA-IR index of Model group was 12.5, that of Experimental group A was 11.3, that of Experimental group B was 10.1, and that of Experimental group C was 9.6. The data show that the HMOA-IR index of Experimental group C decreases by 30.2% compared with the Model group. By ELISA, we detected the contents of TC and TG in serum, and found that compared with the Model group, Experimental groups A, B and C could significantly reduce the contents of TC and TG in serum, among which Experimental group C had the greatest reduction, indicating that Experimental group C (Ergothioneine+Pyrroloquinoline quinone+Oenothera biennis+inositol) has the best effect.

The Example proves that the composition comprising EGT and the method of the invention have a positive regulating effect on the sex hormone levels of female rats, which is beneficial to improving polycystic ovary syndrome and protecting normal functions of ovaries.

The inventors found that the composition comprising EGT and the method of the present invention have a positive regulatory effect on ovarian health, and can significantly promote follicular development, increase the number of follicles, improve the quality of follicles, improve AMH level, and protect or improve ovarian function in mammals, such as delay or improve premature ovarian failure or aging, improve polycystic ovary syndrome, relieve dysfunctional uterine bleeding, delay menopause and/or alleviate menopausal symptoms.

Although specific embodiments and examples of the present disclosure have been illustrated herein, it will be appreciated by those skilled in the art that any modifications and variations can be made without departing from the spirit of the present disclosure. The examples and illustrations above are not intended to limit the scope of the present disclosure. Any combination of embodiments of the present disclosure, along with any obvious their extension or analogs, are within the scope of the present disclosure. Further, it is intended that the present disclosure encompass any arrangement, which is calculated to achieve that same purpose, and all such variations and modifications fall within the scope of the appended claims.

What is claimed is:

1. A method for protecting or improving ovarian function in a mammal, wherein the method comprises administering to the mammal a composition comprising an effective amount of ergothioneine or a pharmaceutically acceptable salt, acid, ester, analog or derivative thereof.

2. The method of claim 1, wherein the composition further comprises an effective amount of Pyrroloquinoline quinone or a pharmaceutically acceptable salt, acid, ester, analog or derivative thereof.

3. The method of claim 1, wherein the composition further comprises an effective amount of Oenothera biennis extract.

4. The method of claim 1, wherein the composition further comprises an effective amount of inositol.

5. The method of claim 1, wherein the protecting or improving ovarian function in a mammal includes promoting follicular development, increasing the number of follicles or improving the quality of follicles.

6. The method of claim 1, wherein the method is used for delaying or improving premature ovarian failure or aging, improving polycystic ovary syndrome, relieving dysfunctional uterine bleeding, delaying menopause and/or relieving menopausal symptoms.

7. The method of claim 1, wherein the composition is formulated as suppository, tablet, pill, granule, powder, film, injection, capsule, aerosol, elixir, tincture, tonic, solution, liquid suspension or syrup.

8. The method of claim 1, wherein the composition is prepared into food, beverage, nutritional supplement, animal feed.

9. The method of claim 1, wherein the composition is administered by oral administration, intravenous injection, intramuscular injection, intraperitoneal or sublingual administration.

10. The method of claim 1, wherein the ergothioneine or the pharmaceutically acceptable salt, acid, ester, analog or derivative thereof is formulated to be administered in an amount of 1-1500 mg per day.

11. The method of claim 2, wherein the Pyrroloquinoline quinone or the pharmaceutically acceptable salt, acid, ester, analog or derivative thereof is formulated to be administered in an amount of 1-1000 mg per day.

12. The method of claim 3, wherein the Oenothera biennis extract is formulated to be administered in an amount of 10-2000 mg per day.

13. The method of claim 4, wherein the inositol is formulated to be administered in an amount of 10-3000 mg per day.
